(19) United States
(12) Patent Application Publication
(10) Pub. No.: US 2020/0101103 A1
(43) Pub. Date: Apr. 2, 2020

(54) REINFORCEMENT OF MUCUS BARRIER PROPERTIES

(71) Applicant: CIRQLE BIOMEDICAL CONTRACEPTION IVS, Copenhagen N (DK)

(72) Inventor: Thomas CROUZIER, Sundbyberg (SE)

(21) Appl. No.: 16/496,921

(22) PCT Filed: Apr. 6, 2018
(86) PCT No.: PCT/EP2018/058922

§ 371 Date: Sep. 23, 2019

(30) Foreign Application Priority Data
Apr. 7, 2017 (EP) 17165525.1

(51) Int. Cl. A61K 31/722; A61K 31/785; A61K 31/717; A61K 9/00; A61K 9/06

(52) U.S. Cl. CPC A61K 31/722; A61K 31/785; A61K 9/06; A61K 9/0034; A61K 31/717

(57) ABSTRACT

A composition or a contraceptive composition comprising a mucoadhesive polymer in a physiologically acceptable carrier, wherein the mucoadhesive polymer consists of 4 to 20 monomers units linked to each other via ether, ester or amide bonds, which monomer units are selected from the list consisting of amino functionalised C6 sugars, C6 sugars, amino functionalised C5 sugars, C5 sugars, amino acids, fatty acid derivatised C6 sugars and fatty acid derivatised C5 sugars and its uses thereof.

### TECHNICAL FIELD

The present invention relates to a composition comprising a mucoadhesive polymer and a physiologically acceptable gelling agent, and its uses in therapy or contraception. The mucoadhesive polymer can crosslink the mucus layer without aggregating the mucus.

### BACKGROUND OF THE INVENTION

There are over 400 square meters of epithelial surfaces hidden within the body of a human being, including the lung, gastrointestinal tract, and the female reproductive tract. The wet epithelial surfaces rely on a mucus gel for protection from dehydration, shear stress, and infections. Besides water, mucus mainly contains mucin biopolymers mixed with proteins, lipids, and salts. Mucins are large glycoproteins, which consist of an extended central protein core densely decorated with oligosaccharides that can account for up to 50% of the molecule's molecular weight. Mucins have a central role in this protective function, creating a size exclusion and affinity-based selective filter, preventing many deleterious molecules from reaching the epithelial surface.

In certain circumstances however, the mucus gel can fail to properly protect the epithelium. For instance, dry eye and dry mouth diseases affect at least 8% of the population. The symptoms can be severely discomforting because of the loss of hydration and lubrication of these surfaces, but also health threatening because of the increased risk of infections. Inflammatory bowel diseases such as Crohn's disease or ulcerative colitis are also linked with a failure of the mucin gel to properly shield the epithelial surface from commensal or pathogenic bacteria. The access of bacteria to the epithelium triggers an inflammatory cycle that is challenging to halt.

Mucoadhesive polymers have been used for drug delivery due to their adhesive properties. For instance, they have been used to deliver drugs to inflammation sites. Mucoadhesive polymers are typically assembled into materials or a gel alongside a drug, the intent being to concentrate the drug at the surface of the mucus layer and improve drug delivery.

WO2004069230 relates to pharmaceutical compositions containing a physiologically active agent, i.e. a drug, and a release sustaining or mucoadhesive agent e.g. chitosan, which serves to prolong the release of the active agent from the composition.

Another use for chitosans is in female contraception. CN102895256 relates to a chitosan gel foaming agent suitable for a female contraception and fungicidal effect and a preparation method thereof, and belongs to the technical field of foaming agent production. According to this disclosure, chitosan molecules are trapped in a solid foam matrix in association with Carbomer, which physically prevents sperm passage. It is known that mucoadhesive molecules promote the tightness and thickening of the mucosal tissue or enhance the barrier function, but usage has shown that the mucoadhesive polymers and mucus-penetrating nanoparticles will crosslink and aggregate the mucus. In particular, when in presence of high molecular weight polyacrylic acids, such as Carbomer, the carboxyl functional groups in the acrylic monomers form ionic complexes with the basic amino groups in the chitosan chain, leading to the formation of a highly swollen interpenetrating polymeric networks. Aggregation of the mucus thus results in opening of pores within the mucus and causes a weakening of the barrier properties of the mucus. There is therefore a need for improvement of cross-linking the mucus without aggregation.

### SUMMARY

With this background it is therefore an object of the present innovation to provide a mucoadhesive polymer which can crosslink the mucus layer without aggregating the mucus. It was surprisingly found by the inventor that an enhanced effect was achieved by a mucoadhesive polymer consisting of 4 to 20 monomer units linked to each other via ether, ester or amide bonds, which monomer units are selected from the list consisting of amino functionalised C6 sugars, C6 sugars, amino functionalised C5 sugars, C5 sugars, amino acids, fatty acid derivatised C6 sugars and fatty acid derivatised C5 sugars, wherein at least 50% of the monomer units of the mucoadhesive polymer comprise an amino group or at least 50% of the monomer units of the mucoadhesive polymer are selected from the list consisting of alanine, methionine, cysteine, phenylalanine, leucine, valine, isoleucine, fatty acid derivatised C6 sugars and fatty acid derivatised C5 sugars. In a first aspect the invention relates to a composition comprising the mucoadhesive polymer defined above and a physiologically acceptable gelling agent. In another aspect the invention relates to a composition comprising a mucoadhesive polymer as defined above and a physiologically acceptable gelling agent for use in therapy, e.g. for the treatment of lesions of the mucus membrane. In a further aspect, the invention relates to a contraceptive composition comprising the mucoadhesive polymer defined above and a physiologically acceptable gelling agent. In another aspect the invention relates to a kit of parts comprising a composition comprising the mucoadhesive polymer defined above, a physiologically acceptable gelling agent, and an applicator.

The small size of the polymer advantageously allows the molecule to diffuse inside the mucus. The improved diffusion of the mucoadhesive polymer into the mucus membrane allows that it can crosslink the mucus layer over a large thickness, without aggregating the mucus. The small mucoadhesive polymers complex to the mucus thereby blocking the pores of the network and reinforcing its barrier properties. Moreover, the small sized polymers are generally more soluble in conditions appropriate for delivery to a mucus membrane of a subject compared to polymers of larger sizes. Thereby, the mucoadhesive polymer can be delivered more efficiently to the mucus membrane, which in turn allows a stronger, and thus more effective, cross-linking than is available using larger mucoadhesive polymer molecules. The mucoadhesive polymer may be either generally cationic, e.g. with at least 50% of the monomers having positively charged amino group, or hydrophobic, e.g. with at least 50% of the monomers having a hydrophobic side chain. The effective duration of the cross-linked mucus is determined by the biological turnover time of the mucus which may vary for the different organs of the body, e.g. eyes, respiratory tracts, mouth, or genital tracts. The cross-linking time may be 1, 2, 3, 4, 5, 6, 7, 8, 9, 10 hours, 1, 2, 3 day(s) or even up to 10 days.

The mucoadhesive polymer therefore provides a more reliable barrier effect which prevents cells and microorganisms such as bacteria, viruses and spermatozoa to penetrate the crosslinked mucus and diffuse into the mucosa membrane. One aspect of the invention is, therefore, a composition, e.g. a contraceptive composition, for preventing pregnancy and/or sexual transmitted infections (STI).

By combination with a physiologically acceptable gelling agent, the contact area between the composition and the mucus is maximised. Increased contact area may help ensure that a maximum amount of mucoadhesive polymer can diffuse into the mucus layer and modify its properties. Also contributing the increased diffusion is a high density of the composition. By having a high composition density, preferably similar to that of water, such as in a semi-solid gel, the applied composition is able to change shape and envelope the full surface of the cervical entrance.

Moreover, the invention provides a contraceptive composition free from hormones or chemicals that have undesired side effects. Undesired effects may include emboli, migraine, or minor side effects such as influencing the menstrual cycle. The effective time of the mucoadhesive polymer according to the invention is determined by the turnover of mucus which means that the contraceptive effect is temporary. The contraception dissolves after the effective time and the fertility is unaffected. The time of sufficient contraception is affected by several factors such as the biological turnover of the mucus, the concentration of mucoadhesive polymer etc., and last for a period of time, such as 1, 2, 3, 4, 5, 6, 7, 8, 9, 10 hours, 1, 2, 3 day(s) or even up to 10 days which is a sufficient time to block sperm cells from entering the cervix. By blocking the sperm from entering the cervix, the acidic environment of the vagina will reduce the motility of the sperm and weaken the sperm leaving them unable to fertilise an egg. Under natural conditions the sperm cells will need to enter the cervix within minutes to survive. The full contraceptive effect is gained from a single application, which means that non-coherent use of the contraceptive composition gives the same protection as coherent use.

The full contraceptive effect is gained from a single application, which means that temporary contact of the cervical mucus with the contraceptive composition gives the same full protection as the continued contact with the cervical mucus.

The mucoadhesive polymer consists of 4 to 20 monomer units linked to each other via ether, ester or amide bonds. Furthermore, polysaccharides monomers can be linked via ether, ester and/or acetal bonds. In one embodiment of the invention at least 50% of the monomers of the mucoadhesive polymer comprise an amino group. In another embodiment of the invention at least 50% of the monomers of the mucoadhesive polymer comprise a hydrophobic group. The amino groups make the mucoadhesive polymer basic, which is advantageous for their binding to the mucus membrane. The basic amino groups particularly provide a more efficient cross-linking. In addition, when at least 50% of the monomers of the mucoadhesive polymer comprise a hydrophobic group, the mucoadhesive polymer can also adhere to and diffuse into the mucus membrane to cross-link the mucus membrane and without aggregating the mucus.

In the context of the invention an amino group is —NH₂, where one or both hydrogen atoms may be substituted with a group R, or the amino group may be a quaternary amino group with 3 R groups. R may be selected from C₁₋₄ alkyls, optionally substituted with one or more —OH, —SH or —NH₂, and when more than one R is present on the same nitrogen atoms these made be the same or different. As long as R has 4 or fewer carbon atoms, in particular when hydrogen atoms of R are substituted by one or more —OH, —SH or —NH₂, the amino groups are generally basic. Longer alkyl chains, e.g. having 5 or more carbon atoms, tend to mask the basicity of the amino group, and amino groups carrying alkyls of 5 or more carbon atoms are not counted as amino groups in the context of the invention. Likewise, the sugar may also comprise an amide group, e.g. —CONHCH₃, or —NHCHO, but such groups are not counted as amino groups in the context of the invention.

The mucoadhesive polymer may be a polysaccharide where C6 or C5 sugars are linked to each other via ether bonds. The monomers of C6 and C5 sugars may be linked via any ether bond, e.g. C1 and C4 of two adjacent C6 sugars may be linked, or C1 and C6 of two adjacent C6 sugars may be linked. In particular, when the monomer is a C6 sugar, e.g. glucose, the monomers, e.g. glucose monomers, may be linked via β(1-4) linkages. In an embodiment the mucoadhesive polymer consists of 4 to 20 β(1-4) linked glucose monomers where at least 50% of the glucose monomers comprise a amino group, e.g. —NH₂. The amino group may be linked to any carbon atom of the glucose monomers, e.g. C2 or C3. In the amino group one or both hydrogen atoms may be substituted with a group R, which may be selected from C₁₋₄ alkyls, optionally substituted with one or more —OH, —SH or —NH₂.

In a preferred embodiment the mucoadhesive polymer is a chitosan where at least 50% of the glucose monomers have an —NH₂ group, and where fewer than 20% of the glucose monomers have a —CONHCH₃ group. The chitosan may also be referred to as at least 50% deacetylated. Further specific embodiments of the mucoadhesive polymer comprise cationised dialdehyde cellulose (DAC), cationic hydroxyethyl cellulose, chitosan, chitosan-trimethyl, chitosan-thioglycolic acid, chitosan-iminothiolane or chitosan-thioethylamidine.

By cationised dialdehyde cellulose and cationic hydroxyethyl cellulose, it is understood dialdehyde or hydroxymethyl cellulose where at least 50% of the glucose monomers are aminated with a cationic amine, said polysaccharides thus being cationic.

In an embodiment of the invention at least 50% of the monomer units are sugar monomers, which are derivatised with a fatty acid. In the context of the invention, the fatty acids are considered to be hydrophobic groups. Any fatty acid is appropriate for the invention, although the fatty acid is preferably a naturally occurring and biologically degradable. Preferred fatty acids have 6 to 22 carbon atoms, in particular, an even number of carbon atoms, and the fatty acids may comprise one or more double bonds. The fatty acid is preferably linked to the sugar monomer via an ester bond.

In a further embodiment of the invention the mucoadhesive polymer is a peptide molecule of a length of 4 to 20 amino acids, which are linked via amide bonds. When the mucoadhesive polymer comprises amino acids, any amino acid may be included as long as at least 50% of the amino acids carry a basic group, or as long as at least 50% of the amino acids carry a hydrophobic group as appropriate. The mucoadhesive polymer is not limited to naturally occurring amino acids, but it is preferred that the amino acids are non-toxic and tolerated by the subject. It is preferred that the mucoadhesive polymer does not comprise D-amino acids but that any amino acid contained in the mucoadhesive polymer is an L-amino acid. In general, the following amino acids are considered basic: arginine, lysine, histidine, ornithine, and β-alanine, and in an embodiment the mucoadhesive polymer is a polypeptide of 4 to 20 amino acids, wherein at least 50% of the amino acids are selected from the list consisting of arginine, lysine, histidine, ornithine, and β-alanine. The remaining amino acids may be selected from any amino acids, e.g. any of the 20 amino acids defined from the genetic code, but in particular glycine, serine, threonine, asparagine, and glutamine. Specific embodiments of the mucoadhesive polymer comprise poly-lysine, poly-orthinine and poly-arginine. The advantage of using basic amino acids is that they have a good solubility in aqueous solutions.

In another embodiment the mucoadhesive polymer is a peptide molecule of a length of 4 to 20 amino acids wherein at least 50% of the amino acids carry a hydrophobic group, which amino acids are selected from the list consisting of: alanine, methionine, cysteine, phenylalanine, leucine, valine, and isoleucine. The remaining amino acids may be selected from the list consisting of: glycine, serine, threonine, asparagine, and glutamine. In one embodiment, the mucoadhesive polymer comprises amino acids, and at least 50% of the amino acids are selected from the group consisting of arginine, lysine, histidine, ornithine, and β-alanine, or 50% of the amino acids carry a hydrophobic group and are selected from the group consisting of arginine, lysine, histidine, ornithine, and β-alanine. It is advantageous to use amino acids or hydrophobic amino acids since they are biodegradable, the protein-peptide interactions between the mucus proteins and the polymer may enhance muco-adhesion. Furthermore, the amino acid polymers may be produced recombinantly using bacteria or synthetically.

In a further embodiment the mucoadhesive polymer comprises both sugar monomers, e.g. C6 and/or C5 sugar monomers, and amino acids, if at least 50% of the monomers are basic, e.g. carry an amino group, or at least 50% of the monomers are hydrophobic, e.g. carry a hydrophobic group.

In another embodiment the polymer consists of 20 or preferably 10 or 8 monomer units, which ensures that the polymer is small enough to diffuse deep into the mucus gel and large enough to form a tight cross-linking network. By tight is meant impermeable to microorganism or sperm cells.

In a further embodiment, the mucoadhesive polymer is selected from polymers with a low molecular weight, which should have a degree of polymerisation (DP) providing a molecular weight in the range of 0.5 to around 5 kDa, which ensures that the mucoadhesive polymer forms stable complexes with the mucus. In a preferred embodiment the mucoadhesive polymer is chitosan and the preferred DP of the chitosan provides a molecular weight in the range of 0.5 to around 3.5 kDa, more preferrably in the range of 0.7-2 kDa, most preferred about 1.5 kDa.

In another aspect, the invention is a kit of parts which comprises the composition comprising the mucoadhesive polymer and the physiologically acceptable gelling agent, e.g. a contraceptive composition as described above, and an applicator. In one embodiment, the applicator is a delivery device similar to what is known from tampons utilised by a method, where the applicator comprising the composition comprising the mucoadhesive polymer and the physiologically acceptable gelling agent, e.g. a contraceptive composition, as described above, in the form of a gel, is inserted in the vagina. The gel is deployed from the applicator and the gel is applied to the cervical mucus and the mucus is crosslinked by the mucoadhesive polymer. In an embodiment, the applicator is a container, which contains the contraceptive composition, e.g. as a contraceptive composition, and which can be emptied by an emptying mechanism. In another embodiment, the container resembles a balloon, which in a first condition contains the composition and in a second condition is removed to administer the composition. In yet another embodiment the kit comprises a mucoadhesive polymer and the physiologically acceptable gelling agent in a composition for treating mucosal lesions, and an applicator where the applicator is a container from which the composition is released as a liquid or drops.

### FIGURES

FIG. 1. Schematic drawing of mucin gel drops in a solution of chitosan and Cryo-SEM image of the surface of the complexes for chitosan DP8, DP52 and DP100, respectively.

FIG. 2. Bar chart of the quantification of the amount of chitosans engaged in the complexation of purified mucin drops.

FIG. 3. Bar chart showing the diffusion of fluorescently labelled dextrans in mucin drop/chitosan complexes.

FIG. 4. Histology images of HT29-MTX culture cultivated on porous membranes. (A) Young, 7-day old cultures revealed multilayer structures. (B) These become polarised and coated with a layer of adherent mucus (stain in blue) after 30 days of culture. (C) Bar chart of the metabolic activity of young HT29-MTX cells after exposure to solutions of chitosan at various concentration. (D) Bar chart of the metabolic activity of mature HT29-MTX after exposure to a 5 mg/mL chitosan solution.

FIG. 5. Graphs showing the output from flow cytometry of HT29-MTX cells exposed to fluorescently labelled dextran (column A) or cholera toxin subunit B (column B). The three conditions tested are HT29-MTX grown for 30 with a mucus layer (line I), HT29-MTX grown for 7 days without a mucus layer and treated with the chitosans (line II), and HT29-MTX grown for 30 days with a mucus layer and treated with chitosans (line III).

FIG. 6. (A) Bar chart showing the quantification of the amount of chitosans engaged in the complex to mature HT29-MTX cultures covered with a mucus layer. Confocal images of HT29-MTX cell layer and the chitosan-FITC deposition on top along with a cross section view for all three chitosans (B) DP8, (B′) DP52, and (B″) DP100.

FIG. 7. Light Microscopy (10x objective) images taken after 3 min, 12 min 30, and 50 min of contact between mucoadhesive amino acid polymers and mucin gels.

FIG. 8. Phase microscopy images of porcine gastric mucin complexed or not complexed with chitosan placed in a sperm solution.

FIG. 9. A) Macroscopic image of a 4 μl mucin gel drop into the chitosan solution. B) Confocal fluorescence image of the border of the complex using fluorescein-labelled chitosan.

FIG. 10. Fluorescence intensity measurement of chitosan oligomers penetration in capillary containing ovulatory human cervical mucus.

FIG. 11. Sperm penetration data for two repeats on human cervical mucus measured in number of spermatozoa per field and penetration distance.

FIG. 12. Chitosan oligomer toxicity towards sperm cells, represented by loss of Progressive motility (%), motility (%), and Velocity (pm/s).

### DETAILED DESCRIPTION

The subject of the present invention relates to a composition comprising a mucoadhesive polymer and a physiologically acceptable gelling agent, wherein the mucoadhesive polymer consists of 4 to 20 monomer units linked to each other via ether, ester or amide bonds, which monomer units are selected from the list consisting of amino functionalised C6 sugars, C6 sugars, amino functionalised C5 sugars, C5 sugars, amino acids, fatty acid derivatised C6 sugars and fatty acid derivatised C5 sugars, wherein at least 50% of the monomer units of the mucoadhesive polymer comprise an amino group or at least 50% of the monomer units of the mucoadhesive polymer are selected from the group consisting of alanine, methionine, cysteine, phenylalanine, leucine, valine, isoleucine, fatty acid derivatised C6 sugars and fatty acid derivatised C5 sugars. The mucoadhesive polymer may be used as a contraceptive, and in another aspect the invention relates to a contraceptive composition with the mucoadhesive polymer and the gelling agent.

In another aspect of the invention, the composition comprising a mucoadhesive polymer and an physiologically acceptable gelling agent is for use in therapy, the mucoadhesive polymer consisting of 4 to 20 monomer units linked to each other via ether, ester or amide bonds, which monomer units are selected from the list consisting of amino functionalised C6 sugars, C6 sugars, amino functionalised C5 sugars, C5 sugars, amino acids, fatty acid derivatised C6 sugars and fatty acid derivatised C5 sugars, wherein at least 50% of the monomer units of the mucoadhesive polymer comprise an amino group or at least 50% of the monomer units of the mucoadhesive polymer are selected from the list consisting of alanine, methionine, cysteine, phenylalanine, leucine, valine, isoleucine, fatty acid derivatised C6 sugars and fatty acid derivatised C5 sugars.

The composition comprising the mucoadhesive polymer and a physiological acceptable gelling agent may be part of a kit further comprising an applicator. The applicator may be used to apply the composition to the surface of e.g. the cervix.

The mucoadhesive polymer may be administered in a physiologically acceptable carrier, which ensures that the mucoadhesive polymer is soluble in the conditions where it is used and ensures that the mucoadhesive polymer is evenly distributed in the target area. Here evenly distributed means that the targeted mucus area is subjected to at least a minimum amount of composition, determinable to the skilled person, with enough mucoadhesive polymer to diffuse into the mucus and reinforce the mucus barrier.

By physiological acceptable carrier is meant a non-toxic compound, which in an effective dose, is neither chemically nor physically toxic to a human or animal organism or a biological process.

In an embodiment the pharmaceutical acceptable carrier is water, DMSO, saline, or a combination thereof.

Mucoadhesion is here described as the interfacial forces that hold together two biological materials, such as the attractive forces between a biological material and mucus or a mucus membrane. By mucoadhesive polymer, is therefore meant a polymer which has attractive force towards mucus or a mucus membrane.

Mucus is the protective cover of all epithelial surfaces, which keep the epithelial layer moist and prevent microorganism from invading the epithelium. A natural protective effect is achieved because the mucus traps microorganisms and facilitates their distal transport. When mentioning the barrier effect achieved by the mucoadhesive polymer, it is the reinforcement of the mucus due to cross-linking of the polymer. The effect of the reinforced barrier is based on the tightness of the cross-linked mucus which stops diffusion, and how long the mucus is reinforced by the complexed mucoadhesive polymer. The latter is determined by the natural turnover of mucus cells from the mucosa, which removes the mucus comprising the cross-linked polymer.

The mucus layer on the mucosa differs in thickness and is based on different biological factors, such as e.g. segment of the body, animal species, age or outbreak of diseases, and the thickness which is affected by the mucoadhesive polymer is therefore dependent on these factors and could vary from a few microns to hundreds of microns. Which thickness of complexed mucus and therefore the barrier effect which is required to prevent a foreign object from diffusing into the mucosa membrane is depending on the use of the mucoadhesive polymer. One thickness of barrier layer might be impermeable to relatively large cells, such as spermatozoa, whereas an even tighter barrier layer may be required to be impermeable to bacteria or viruses or other microorganism or infections.

The small mucoadhesive polymer consists of 4-20 monomer units, which are linked to each other via ether, ester or amide bonds. The monomer units are selected from the list consisting of amino functionalised C6 sugars, C6 sugars, amino functionalised C5 sugars, C5 sugars, amino acids, fatty acid derivatised C6 sugars and fatty acid derivatised C5 sugars, wherein at least 50% of the monomer units of the mucoadhesive polymer comprise an amino group or at least 50% of the monomer units of the mucoadhesive polymer are selected from the list consisting of alanine, methionine, cysteine, phenylalanine, leucine, valine, isoleucine, fatty acid derivatised C6 sugars and fatty acid derivatised C5 sugars.

A small polymer is here a polymer with a low degree of polymerisation (DP), such as 20 monomers or below, preferably in the range of 5 to 20, such as a DP of 8, and with a molecular weight below 5 kDa, preferably in the range of 0.7 to 2 kDa, such as 1.5 kDa.

The small size polymer ensures that the polymer is soluble in the conditions used, and that the polymer can diffuse through the pores of mucus and form a thick and tight barrier.

The mucoadhesive polymer should be stable in the environment of the targeted mucosa, which can range from a low pH in e.g. stomach or the female abdomen, to a neutral or weak basic pH in cells. The pH range where the mucoadhesive polymer is stable is therefore in the range of 1-8. Dependent on the pH environment, different types and sizes of polymers may be used. A DP8 chitosan is for instance soluble at basic pH, while a DP52 chitosan and DP 100 are only soluble at pH <6.

The diffusion of the polymer occurs when the mucoadhesive polymer and a physiologically acceptable gelling agent adheres to the mucus. The use of the mucoadhesive polymers in therapy is possible due to its low degree of polymerisation and degree of acetylation, which gives good mucoadhesion. This allows the polymer to diffuse into the mucus and temporarily block the pores of the mucus. This occurs due to a temporary crosslinking effect of the mucus, which is controlled by the normal turnover of mucus and the biodegradability of the mucoadhesive polymer. The effective cross-linking time can therefore be adjusted by subjecting the mucus to different concentrations of the mucoadhesive polymer such as for example a concentration of 1-100 mg/mL, such as in the range of 5 mg/mL.

The mucoadhesive polymer will, due to its adhesive properties and small size, penetrate the mucus and diffuse into the surface of the mucus to form a thick layer. The small mucoadhesive polymer will then complex to the mucus and thereby block the pores of the network, providing a reinforced-barrier property to the mucus. When the mucus is reinforced, it is impermeable to particles, and prevents passage e.g. externally induced liquids, particles and cells, such as spermatozoa. The complex formed in the mucus could be targeted against a certain size of cells, and thereby be impermeable to virions of the range of 20-30 nm in size, mycoplasma in the range of 0.3 microns, bacteria in the range of 0.5 to 5 microns, or spermatozoa in the range of 3 microns.

The composition of the invention comprising a mucoadhesive polymer and a pharmaceutically acceptable gelling agent is a contraceptive agent, since the treated mucus will be temporarily impermeable to spermatozoa. The contraceptive effect in connection with the present invention means a reversible and temporary prevention of pregnancy due a non-surgical, hormone-free and non-coherent barrier effect achieved by a single use, meaning that the contraceptive effect is achieved by one application, and does not require a concentration to be developed over a period of time, as it does with pills.

The composition of the invention comprising a mucoadhesive polymer and a pharmaceutically acceptable gelling agent is a contraceptive agent since the treated mucus will be temporarily impermeable to spermatozoa. The contraceptive effect used in connection with the present invention means a reversible and temporary prevention of pregnancy due a non-surgical, hormone-free, temporary barrier that is achieved within minutes of contact of the cervical mucus with the solution and is stable with or without further action of the contraceptive agent for hours. The effect is achieved by a single use, meaning that the contraceptive effect is achieved by one application, and does not require a concentration to be maintained over a period of time.

By temporary effect is implied that, when applied to the mucus, the effect of the mucoadhesive polymer is reversible. The rate at which the reversion will occur is determined by the amount of polymer diffused into the mucus and by the biological turnover of the mucus itself.

Lesions means, in the context of the invention, any damage or undesired change in tissue structure or composition. This may occur anywhere in a human and or animal body, and include soft-tissue lesions, skin lesions, intestinal lesions and any lesions to mucosal tissue, such as the lungs or other organs or abdomen tissue such as but not limited to, the cervix, vagina and/or uterine.

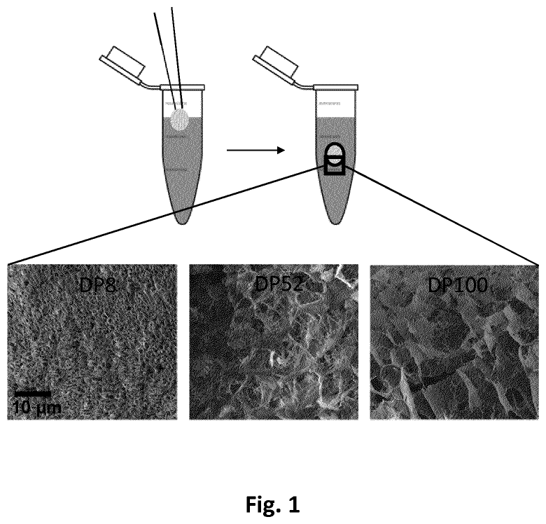

FIG. 1 is a schematic drawing of mucin gel drops in a solution of chitosan and Cryo-SEM images of the surface of the complexes for chitosan DP8, DP52 and DP100, respectively.

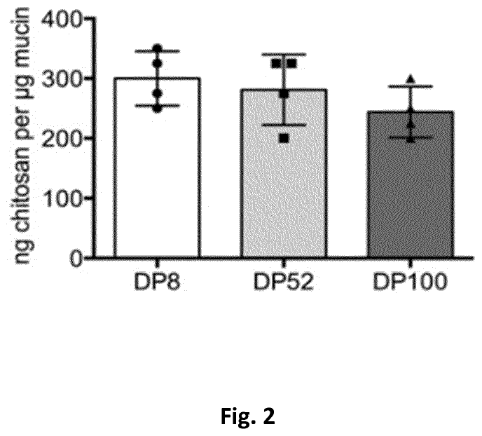

FIG. 2 shows a quantification of the amount of chitosans engaged in the complexation of purified mucin drops.

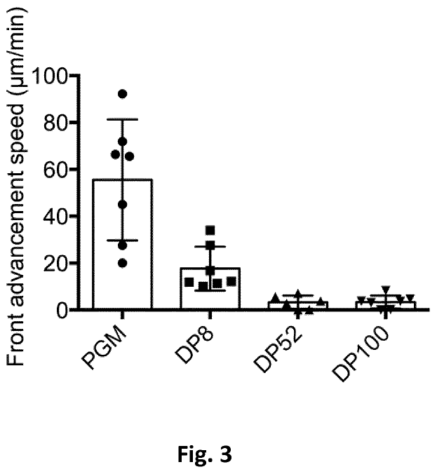

FIG. 3 illustrates the diffusion of fluorescently labelled dextrans in mucin drop/chitosan complexes. (D) Diffusion front advancement speed for dextran 70 KDa.

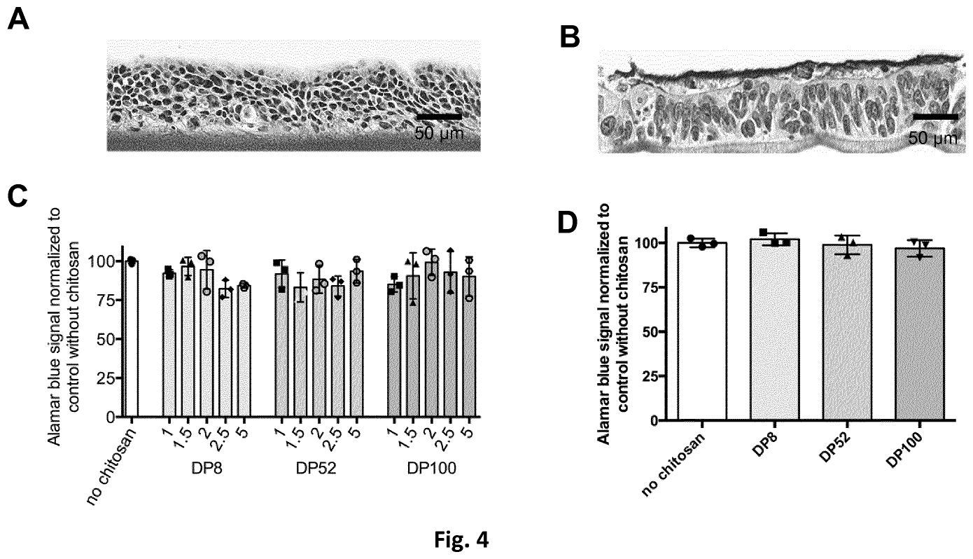

FIG. 4 shows the toxicity of chitosan solutions on HT29-MTX cells. Histology of HT29-MTX culture cultivated on porous membranes. (A) 7-day old cultures revealed multilayer structures. (B) These become polarised and coated with a layer of adherent mucus (stain in blue) after 30 days of culture. (C) Metabolic activity of 7-day HT29-MTX cells after exposure to solutions of chitosan at various concentrations. (D) Metabolic activity of mature HT29-MTX after exposure to a 5 mg/mL chitosan solution.

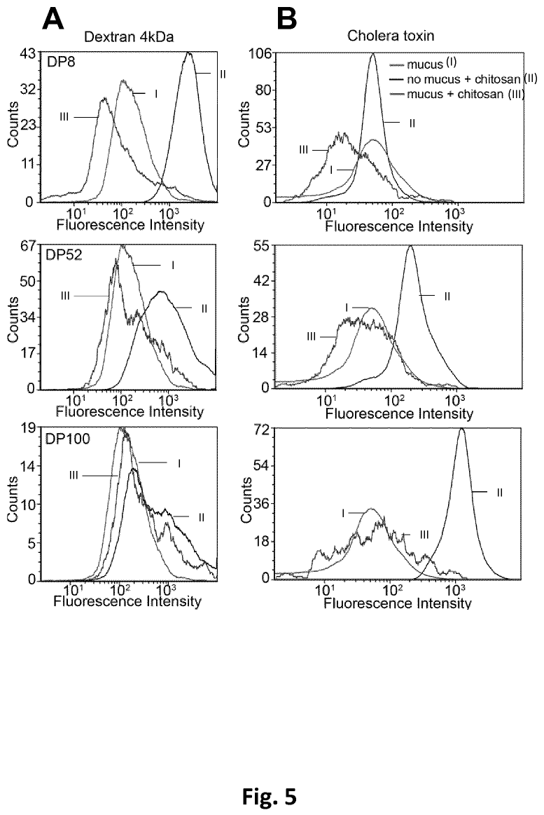

FIG. 5 shows the results from flow cytometry analysis of HT29-MTX cells exposed to fluorescently-labelled dextran (column A) or cholera toxin subunit B (column B). The three conditions tested are HT29-MTX grown for 30 days with a mucus layer (line I), HT29-MTX grown for 7 days without a mucus layer and treated with the chitosans (line II), and HT29-MTX grown for 30 days with a mucus layer and treated with chitosans (line III).

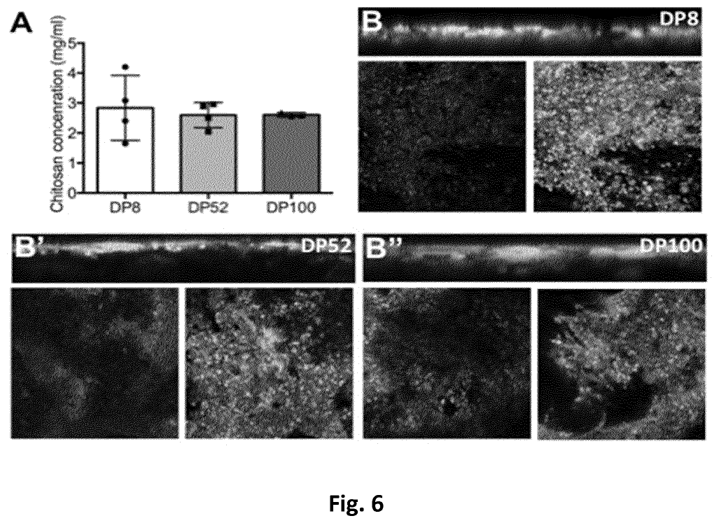

FIG. 6 shows the results from quantification of the amount of chitosans engaged in the complex to mature HT29-MTX cultures covered with a mucus layer. Confocal images of HT29-MTX cell layer and the chitosan-FITC deposition on top along with a cross section view for all three chitosans (B) DP8, (B′) DP52, and (B″) DP100.

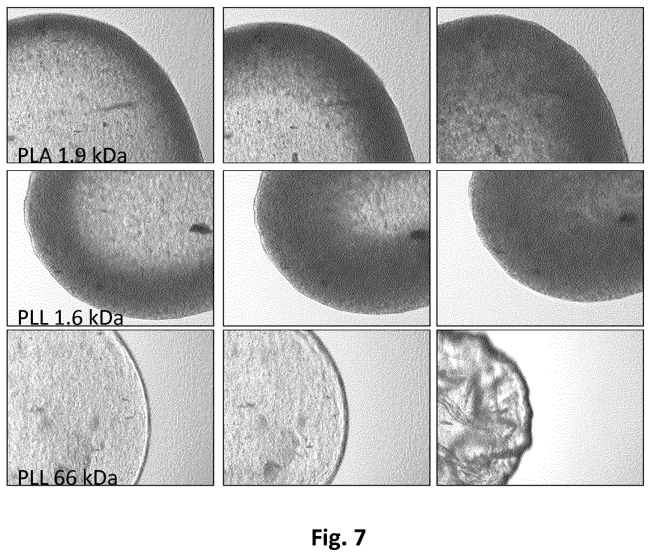

FIG. 7 illustrates the effect of low or high molecular weight muco-adhesive polymers on mucin gels. A 4 μl drop of purified porcine gastric mucin was placed in a solution of poly-L-arginine (PLA, DP 11 corresponding to 1.9 kDa, 5 mg/mL), poly-L-lysine (PLL, DP 11 corresponding to 1.6 kDa, 5 mg/mL), or poly-L-lysine (PLL, DP 400 corresponding to 66 kDa, 5 mg/mL). The drop of mucin was observed under the microscope (10x objective). The three images are taken after 3 min, 12 min 30, and 50 min of contact. The small poly amino acids clearly crosslink the mucin in depth, while the large poly-amino acid polymer changes the structure of the mucins drop and compacts or aggregates it significantly.

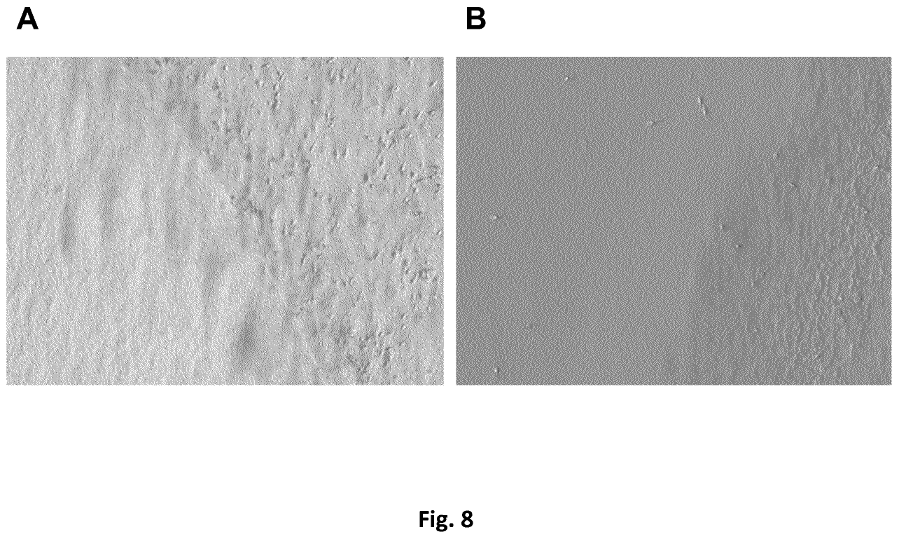

FIG. 8 shows a 4 μl drop of purified porcine gastric mucin complexed using a solution of chitosan (DP8, 5 mg/mL), or not complexed, placed in sperm solution. The images were recorded after 2 minutes. The complexed mucin had no sperm cell penetrating the outer shell, while the non-complexed mucins drop let sperm cell penetrate the gel.

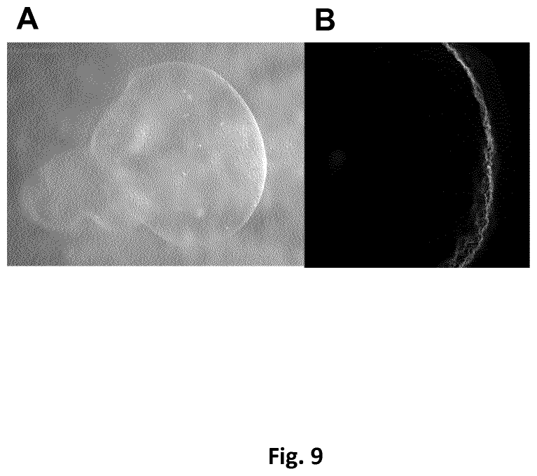

FIG. 9 illustrates the complexation of pig gastric mucin gel (10 mg/ml, pH 6) with high molar mass chitosan (550 kDa, deacetylation degree of 98%) at 2.5 mg/ml pH 5.5 and shows a macroscopic image of a 4 μl mucin gel dropped into the chitosan solution after one hour of complexation (drop size is about 800 μm), and a confocal fluorescence image of the border of the complex using fluorescein-labelled chitosan. The larger chitosan was able to bind but not penetrate the mucus drop.

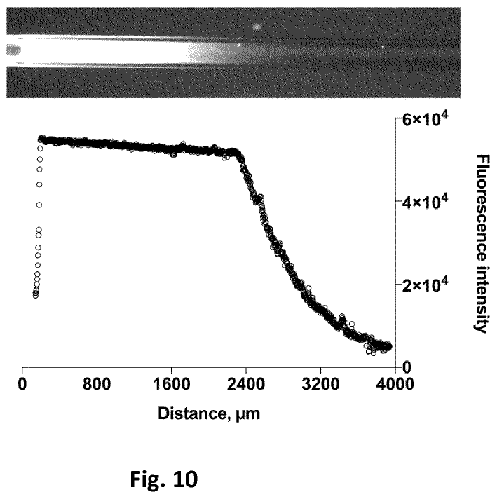

FIG. 10 shows penetration of chitosan oligomers (molar mass of 1.4 kDa, deacetylation degree of 89%) fluorescently labelled with fluorescein into ovulatory human cervical mucus.

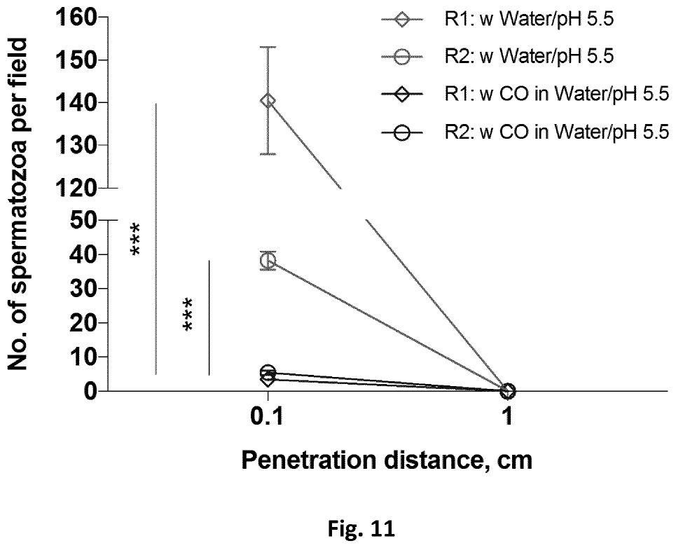

FIG. 11 shows the effect of the addition of oligo-chitosan (CO, molar mass of 1.4 kDa, deacetylation degree of 89%) dissolved in water pH 5.5) on sperm penetration in human cervical mucus.

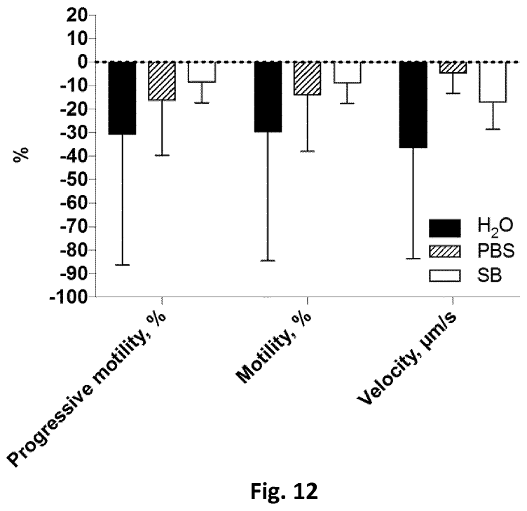

FIG. 12 illustrates chitosan oligomer toxicity towards sperm cells (2.5 mg/mL, molar mass of 1.4 kDa, deacetylation degree of 89%) dissolved in water (H2O), phosphate buffered saline (PBS) or semen buffer (SB).

### EXAMPLES

Materials and Methods.

Pig Gastric Mucin Purification.

Mucins were purified from the mucosa of porcine stomachs following previously published protocol. Commercially available mucins were not chosen for this study because of their known altering biophysical properties, which must be maintained stable for the present study. Briefly, the mucus was gently scraped off the epithelium, diluted 1:5 in water supplemented with 200 mM NaCl, as well as 5 mM benzamidine HCl, 1 mM 2,4′-dibromoacetophenone, 1 mM phenylmethylsulfonyl-fluoride, and 5 mM EDTA. The pH was adjusted to pH 7.4 with NaOH and the mixture added and gently stirred overnight at 4° C. The solution was first centrifuged, then ultracentrifuged to remove cellular and food debris, before being fractionated by preparative size exclusion chromatography. The excluded fraction contained high molecular weight and highly glycosylated molecules, as confirmed by periodic acid Schiff assay. The fractions were pooled, then desalted and concentrated by reversed osmosis using a 100 kDa molecular weight cutoff. Finally, the mucins were flash frozen in liquid nitrogen and lyophilised, and stored at −20° C. until used.

Commercially available pig gastric mucins, fluorescently labelled dextrans (4 kDa and 70 kDa), and fluorescein labelled cholera toxin B subunit were purchased from Sigma-Aldrich.

COS and LMW Chitosan Production and Labeling

Chito-oligosaccharides (COS) and low molecular weight (LMW) chitosans were produced by nitrous acid depolymerisation of commercial chitosan, according to an adapted protocol previously described. Briefly, commercial chitosan (1g; Mn=115 kDa; D=2.3; degree of N-acetylation (DA) <1%; batch 244/020208 supplied by Mahtani Chitosan Ltd., India) was solubilised in 50 mL of water by addition of concentrated HCl. A freshly prepared aqueous solution of NaNO₂ (GlcN/NaNO₂ molar ratio =4, 25 and 75 for DP8, DP52 and DP100 chitosans, respectively) was added and the reaction was stirred for 12 h at room temperature. DP8 chitosan (0.7g, 70% mass yield) was obtained after io neutralisation of the solution by addition of ammonium hydroxide solution until pH ˜8, ultrafiltration (MWCO 500), precipitation in alcohol and drying under vacuum. DP52 (0.8 g, 100% mass yield) and DP100 (0.9 g, 90% mass yield) chitosans were obtained after precipitation of the solution by addition of ammonium hydroxide solution to pH ˜8, several washings with distilled water until neutral pH and freeze-drying. The DP and the number-average molar mass of chitosan were determined by 1H NMR and SEC-MALLS, respectively, as previously described. Before use, the chitosans were dissolved in ultrapure water at 10 mg/mL and the pH was adjusted to 5 using 36% HCl and readjusted if necessary using 2N HCl or 2N NaOH with vortexing or placing on shaker at 4° C.

Chitosans were labelled with fluorescein following an adapted version of a published protocol. Briefly, 4% solution of DP8, DP52, DP100 chitosans were prepared in 2 mL of ultrapure water and the pH adjusted to pH 5.5 with NaOH. 2 mL of methanol were then added to the solution. FITC dissolved in DMSO was added to the mixture at a ratio of 1 fluorescein molecule for every 50 chitosan monomers. The solution was shaken for 2 hours at room temperature, then 4 volumes of ethanol were added to precipitate the chitosan. For the DP8 chitosan the precipitate was obtained by addition of a combination of ethanol and NaOH to reach pH 9. The precipitated chitosans were rinsed with ethanol until free FITC was removed, then lyophilised and stored at −20° C. until used.

Chitosan-Mucin Complexes.

For complexation with chitosans, the pig gastric mucins were first dissolved overnight at 10 mg/ml in ultrapure water. A 4 μl drop was then slowly pipetted in 300 μL of a 5 mg/mL chitosan solution diluted from stock in water. The drop was left to complexation for over 1 hour. Then the drop was removed from the chitosan solution and placed in water until used. For complexation with HT29-MTX epithelial model, the stock chitosans solutions were further diluted to 5 mg/mL using acidified 1× D-PBS (sterile), pH 5.5 and added to cells for one hour.

Barrier Properties of Chitosan/PGM Complexes

The barrier properties of reconstituted mucin gels were assessed using fluorescently labelled dextran (4 kDa and 70 kDa). First, complexes were formed as described above, then the drops were washed in 20 mM HEPES solution (pH 7) for 5 to 30 minutes before being introduced in a solution of 1 mg/ml dextran-FITC in 20 mM HEPES, pH 7. The drops were then imaged about 30 seconds after being introduced to the solution and every minute thereafter using a Zeiss LSM510 fluorescence confocal microscope and a 10× objective. Image analysis of the time lapse was performed using ImageJ. A diffusion front limit was set as being 5% of the fluorescence of the solution outside the mucin drop. The distance of the diffusion front was linearly related to the square root of time and revealed a random motion type of diffusion.

HT29-MTX Cell Culture

Frozen vials of HT29-MTX10-6_CelluloNet N° 566 cells were acquired from CelluloNet biobank BB-0033-00072 facility of SFR Biosciences (UMS3444/US8). Cells were thawed and sub-cultured routinely using basal medium consisting of DMEM/F12 (1:1) (1×)-Glutamax (Sigma Aldrich) supplemented with heat inactivated 10% FBS (Hyclone), 1% Penicillin/Streptomycin (Sigma Aldrich) and 1 mM sodium pyruvate (Sigma Aldrich). Primary expansion of the cells was done in 25-cm² T-flasks (Sarstedt) at 37° C. in a 5% CO₂/95% air atmosphere. For maintenance purposes, cells were passaged weekly using 0.25% trypsin in 0.53 mM EDTA (Sigma Aldrich) after 1× PBS washes and plated at 1:10 dilution in 75-cm² T-flasks (Sarstedt) at 37° C. in a 5% CO₂/95% air atmosphere. The medium was changed every second day in all culture conditions. Cells were cultured until 75% confluence before subculturing. Cells with passaging cycles between 19-35 were used for experiments.

Mucus Production in HT29-MTX Cells

A model mucosal surface was created by growing HT29-MTX cells on 12 mm Transwell supports with 0.4 μm pore polycarbonate membrane inserts (Corning Inc. USA). The cells were seeded at the apical side of the membrane at a density of 2×104 cells in 0.2 ml fresh media and 1 ml media was added to the basolateral compartment. 4 days after seeding, the culture reached confluency and a semi-wet interface was produced by leaving 1 ml of media in the basolateral compartment and 50 μl of media in the apical compartment. To enhance mucus production, 10 mM N-3,5-Difluorophenyl) acetyl-L-alanyl-2 phenylglycine-1,1-dimethylethyl ester (DAPT) (Sigma-Aldrich, Sweden) was added basolateraly and apically for 6 days. Thereafter, the medium in the apical compartment was removed and left empty, while 1 ml medium was left in the basolateral compartment. Medium was replenished every second day for 4 weeks before using the cultures for further experiments. A non-homogenous layer of mucus starts appearing after week-2 of culture and covers most of the surface by week-4.

Metabolic Assays

The effect of the exposure of the chitosan solutions on HT29-MTX cell cultures was evaluated by measuring metabolic activity of both mucus-poor 7-day old cultures grown in media and mucus-rich 30-day culture grown at the air-water interface. The cell culture medium was removed and 100 μl of the oligo-chitosan solutions were added to the apical surface of the 4-week old cultures or to 7-day cell culture previously seeded at 5×10⁴ cells/well in a 24-well plate (Nunc) and cultured for 7 days to confluency. After 1 h of incubation, the chitosan solution was removed from the inserts or the wells and washed three times with sterile D-PBS. Each experimental condition was performed in triplicates. After the treatment conditions were met, the cells were allowed to recover for 24 h. Changes in metabolic activity were measured using a resazurin-based in vitro toxicology assay kit (Alamar blue, Sigma). A 10% Alamar blue working solution was prepared in complete medium with 1% heat inactivated FBS and incubated with the cell cultures in the dark for 3 h at 37° C. in a 5% CO₂/95% air atmosphere. The reduced Alamar blue was aliquoted into a 96-well plate (NUNC) and fluorescent measurements (Exc 545 and Em 590 nm) were taken using the plate reader (BMG-CLARIOstar).

HT29-MTX Histology

Mature and young cells cultured on Transwell membranes were washed with 1× sterile DPBS. The culture inserts were then immersed in 250 uL methanolic Carnoy's solution (Methanol: chloroform: acetic acid (60:30:10 v/v) overnight for mature cultures and 4% paraformaldehyde (PFA) exposure for 30 mins for young cultures was used. The membranes were folded to enclose the mucus layer and protect it. These cultures were then paraffin embedded. Embedding, sectioning, alcian blue staining and slide scanning were performed at the SciLifeLab Tissue Profiling Facility at Uppsala University. The membranes were cut out from the inserts, folded to prevent disturbance to the mucus layer and subsequently embedded in 5% SeaPlaque agarose (Lonza, #50100) and processed for paraffin embedding using standard procedures. Embedded samples were cut in microtome at 4 μm and collected on SuperFrost Plus slides, baked at 55° C. overnight and kept frozen until use. For mucin staining, slides were deparaffinised in xylene and re-hydrated in graded alcohols to distilled water before being treated with 3% acetic acid for 3 minutes at room temperature before being treated with Alcian blue (1%, Sigma Aldrich) for 30 minutes at room temperature. Subsequently, slides were washed in running water for 2 minutes and then rinsed in distilled water before being counterstained in Mayers hematoxylin (01820, Histolab) for 5 minutes using the Autostainer XL (Leica). Slides were dehydrated in graded ethanol and lastly coverslipped (PERTEX, Histolab) using an automated glass coverslipper (CV5030, Leica). The slides were scanned into high-resolution digital images using a 40× objective in an automated scanning system (Aperio XT, Aperio Technologies).

Cryo-SEM Observations

Chitosan complexed mucin drops were washed in water then deposited on an aluminum micro-plate. A detailed explanation of the method followed for Cryo-SEM observation has been described previously. Care was taken to prevent sliding of samples by placing a drop of Tissue-TEK prior to the freezing in liquid nitrogen (LN₂) step. After freezing, the grip-holder and plate containing samples were immediately transferred into the LN₂ cooled cryostat (ALTO 2500 cryo-preparation chamber), a sample preparation chamber attached to the microscope (FEI Strata 235DB dual-beam FIB/SEM) and the surface exposed by fracturing with a precooled blade. The samples were then sublimed at −60° C. for 2 mins. Still under LN2 and high vacuum, the samples were sputter-coated with a continuous stream of Ar gas in the cryo-chamber at 7V under high-vacuum for 40 s. The samples were then transferred into the scanning electron microscope chamber and observations were performed at 10 kV with the chamber at −170° C. Images were acquired at 5000× magnification.

Barrier Property of HT29-MTX Mucus.

Cell culture medium was removed from the apical side of 4-week old HT29-MTX cultures or from young cultures and replaced by 0.1 ml of oligo-chitosan solution. After 1 h, the chitosan solution was removed from the inserts and washed 3 times with 1× D-PBS before adding either 0.1 ml of dextran-FITC or 1 ml of FITC-Cholera-toxin B. Dextran-FITC with (˜4 kDa, Sigma Aldrich) was dissolved in sterile milliQ water at 1 mg/ml, while FITC-Cholera-toxin B (Sigma Aldrich) was dissolved at 50 μg/ml in medium without serum or antibiotics. The plates were incubated at 37° C. in 5% CO₂/95% air for 1 h then washed with 1× D-PBS. The young cells were collected after a 15 min trypsin treatment using 0.5 ml of 0.25% trypsin-EDTA at ° 37° C. in 5% CO₂/95% air. The mature cultures were washed with 1× D-PBS and subsequently washed with a sodium bicarbonate-NaCl buffer (0.1N NaCl buffered to pH 7.4 and containing 0.1 M sodium bicarbonate and 1* 10-3 M DTT) and incubated with 0.25 ml 1 mg/mL DTT for 15 min at 37° C. in 5% CO₂/95% air to break the mucus barrier and subsequently trypsinised similarly to the young cells. Post-trypsin treatment all cells were spun down on a centrifuge (Eppendorf 5417C) at 4° C. for 3 mins at 1700 rcf. The supernatant was discarded and the pellet was resuspended in 0.3 ml of 1% BSA-D-PBS and transferred to sample tubes for flow cytometric analysis (Galios, Beckman Coulter). A viscous gel would assemble in the tube if left for standing for long periods during experiments, thus vigorous vortexing was used to disrupt the clusters immediately before measurements. Data was analysed using DeNovo FCS Express flow cytometry software. All samples were tested in duplicates.

Chitosan Quantification

The chitosan contained in each of the complexes were quantified using fluorescamine, which becomes fluorescent when bound to the primary amines of the chitosan. In a glass vial containing 100 μl of chitosan at 5 mg/ml, 5 PGM-chitosan drops were formed. The concentration of chitosan before and after complexation were compared. To do so, 2 μl of each sample were taken and diluted in 48 μl of a 200 mM MES buffer adjusted to pH 5.5. Then 100 μl of fluorescamine at 2 mg/ml dissolved in DMSO were added and let to react before the fluorescence of each solution was measured using a microplate reader (Clario Star, BMG Labtech) using an excitation wavelength of 390 nm and an emission wavelength of 515 nm. The same technique could not be used to measure the amount of chitosan complexed to the HT29-MTX cell cultures due to the presence of contaminants from the cell culture. Chitosan-FITC solution were exposed to the mucus-covered HT29-MTX cell cultures obtained a previously described. The fluorescence of the solution before and after exposure to the cells was compared to estimate the amount bound to the cell culture.

Visualisation

The distribution of chitosan on the HT29-MTX cell culture was visualised in 4-week old mature HT29-MTX cell cultures. The cells were first labelled using a non-toxic membrane dye (Celltracker DeepRed, Invitrogen) dissolved in media without serum and which was added to the basolateral side for 1 hour at 37° C. in 5% CO₂/95% air. The cells were then exposed to chitosan-FITC solutions for 1 hour as previously described. After removing the chitosan, a drop of 0.5% agarose in D-PBS was added to the apical side to preserve the mucus structures. The cultures were then using a laser scanning confocal microscope (LSM800, Zeiss, germany) using a 20x objective.

### Statistical Analysis

The statistical differences of the data presented in FIGS. 2, 3, 4, and 6 and were tested using an unpaired t test. The data pair were deemed statistically different if the probability of occurrence of the data assuming a normal distribution was found to be lower than 0.05 (p<0.05)

Penetration of chitosan oligomers into ovulatory human cervical mucus Chitosan oligomers (molar mass 1.4 kDa, deacetylation degree 89%) where fluorescently labelled with fluorescein. A 0.3×0.3 mm square capillary was filled with cervical mucus and exposed for 30 minutes to a 5 mg/ml solution of chitosan, pH 5.5. The fluorescence was then measured by fluorescence s microscopy. Chitosan fluorescence could be detected up to 0.4 cm from the capillary edge.

### Example 1

### Chitosan Binds Mucin Gels to Form Insoluble Objects

We first tested whether the three chitosan preparations could bind to mucins. We first immobilised biotinylated mucins on pegylated antifouling surfaces which assured only mucin-chitosan interactions would be recorded. The interaction between chitosans and mucins was measured using quartz crystal microbalance with dissipation (QCM-D), which records changes in weight on the surface of the sensor, as well as the dissipation of an acoustic wave which reflect the mechanical properties of the layer. The QCM-D frequency drop indicates that all chitosan solutions could bind to both the in-lab purified pig gastric mucins (PGM) and the commercially-available bovine submaxillary mucins (BSM) (Table 1). Interestingly the larger chitosan chains (DP52 and DP100) dissociated more from the mucin when washed with buffer than the smaller chitosan (DP8). In all cases, the addition of chitosan also led to an increase in dissipation, which suggest that the complexation of chitosan leads to thicker and more viscoelastic film on the surface. These experiments thus confirmed the mucoadhesive nature of the three chitosans studied herein.

TABLE 1
QCM-D frequency shift and dissipation values for chitosan
interaction with either pig gastric mucins (PGM) or bovine submaxillary
mucin (BSM) attached to passivated pegylated surface.

| | DP8 | | |
|---|---|---|---|
| | chitosan | DP52 | DP100 |
| | (Mn = | chitosan | chitosan |
| | 1.3 kDa) | (Mn = 8.4 kDa) | (Mn = 16.1 kDa) |
| PGM - Frequency | −23.8 | −12.3 | −12.5 |
| (ΔHz) | | | |
| PGM - Dissipation | 3 | 2.9 | 1.6 |
| BSM - Frequency | −18.7 | −12.5 | −15.1 |
| (ΔHz) | | | |
| BSM - Dissipation | 3.1 | 2.9 | 2.3 |

### Example 2

### Chitosan Binds to Mucin Gels and Forms Insoluble Spherical Objects

We then tested the effect of the chitosan-mucin interaction on the structure and barrier properties of three dimensional mucins gels, a configuration which better mimics natural mucus than mucin anchored onto a surface. To improve io the visualisation of the effect that chitosans complexation could induce on mucus gels, we first used a reconstituted mucus model consisting of previously characterised in-lab purified pig gastric mucins. We found that when dropping weak mucin gels (10 mg/ml, pH 6) into a chitosan solution (pH adjusted to 5.5 with HCl, 5 mg/ml), the mucin drop immediately reacted, becoming visibly opaque and forming a capsule within seconds. The mucin gel was thus quickly stabilised, forming an insoluble object that could be easily pipetted and manipulated.

The complexation was stopped after one hour, at which point there was no visible additional opacification of the drops. The diffusion of the chitosan within the drop was visualised by using confocal microscopy. The cross-section of the chitosan-mucin drops revealed that the small DP8 chitosan distributed more homogeneously throughout the mucin drop (FIG. 1). A fraction of the DP52 and DP100 chitosan molecules could also diffuse within the mucin drop, and preferentially accumulated at the periphery (FIG. 1). In all samples, we noted the presence of dark zones which excluded the chitosan. This is likely due to the heterogeneity of purified mucin gels which can contain microdomains of concentrated mucin microgels. The distribution of the chitosan in the drop depends on their ability to freely diffuse through the mucin network. High molecular weight chitosans are more likely to be sterically trapped and chemically bound to the mucin mesh than their smaller counterparts. In addition to size and number of interactions sites, the three chitosans tested here have different physicochemical properties. For instance, the DP8 chitosans are soluble at all pH, whereas DP52 and DP100 are only soluble at pH below 6.2. Although these differences could affect their interaction with mucin gel, it is unclear in what way.

Given the clear evidence for chitosan interaction and diffusion across mucin gels, we tested if the chitosan complexation impacts the structure of the gels. We used CryoSEM to investigate the changes to the mucin gels after complexation. One must keep in mind that the sample preparation for electron microscopy techniques inevitably alters the native structure of the hydrogels. However, differences in the structure of the complexes were observed between the three conditions that were compared (FIG. 1). Mucins complexed with DP8 chitosan resulted in a finer morphology than when complexed with DP52 and DP100 chitosans. In these later conditions, large homogeneous structures were interconnected by thin network of branched membranes. These differences could arise from the nature or density of the individual crosslinks formed by DP8 chitosan compared to those formed by the larger DP52 and DP100 chitosans. While larger chitosans would interact with mucins over large scales, smaller chitosan oligomers could bridge mucins at a much smaller scale resulting in the smaller features structures observed by SEM. These changes in observed gel structure could also be explained by invoking differences in the total amount of chitosan complexed by the mucin drops. However, the quantification of the amount of chitosan revealed no significant differences amongst the three molecules, around 0.3 pg of chitosan per pg of mucins. (FIG. 2).

### Example 3

### Chitosan Complexation Modulates the Barrier Properties of Reconstituted Mucus

Given the structural changes induced by chitosan complexation, we hypothesised that the barrier properties of the gel could also be affected. We thus used mucin-chitosan drops to study the diffusion of fluorescently-labelled dextran into the complexed mucin gel. The drops were immersed in a solution of dextran and the advancement of the diffusion front was followed over time by confocal fluorescence microscopy (FIG. 3). We chose dextrans because of their known limited interaction with mucins and their availability as fluorescein conjugates. We thus mainly probe the size exclusion effect of the mucin gel and not its ability to filter through affinity interactions. The progressions of the dextran molecules of 70 kDa, with a Stokes radius of ˜3.6 nm) were slower in mucin gel that were complexed with any of the chitosans than in un-altered mucin gels (p<0.05). In this system, the smaller DP8 chitosan led to the smallest decrease in the front advancement speed of dextrans (FIG. 3). For larger DP52 and DP100 chitosans, the dextran molecules could hardly progress in the mucin gel complexes at the short times observed here (10 minutes). Put together these results suggest that chitosan complexation of mucin slows the diffusion of dextran through the mucin gel, reinforcing its barrier properties.

### Example 4

### Mucus Producing Model—Effect of Chitosans on HT29-MTX Mucus Secreting Epithelial Cell Model

Although informative, the purified mucin model is a simplification of native mucosal barrier. We next used mucus-secreting HT29-MTX cells cultured at the air water interface to mimic more closely the native mucosal tissue. The mucin gel is thus replaced by a much more complex mucus, which is composed of mucins and other component such as lipids, other proteins, and carbohydrates. The barrier is also structurally different, composed of a loose and a more adherent mucus, as well as cell-membrane tethered layer on the cell surface. The HT29-MTX formed multicellular structures at seven days of culture (FIG. 4A). We confirmed by histology the presence of a 20 μm thick mucus layer after 30 days of culture (FIG. 4B) and verified the presence of two major secreting mucins Muc5AC and Muc5B by mass spectrometry. In contrast, young cell cultures of 1 week of age, did not present the mucus layer and were used as a control condition.

We first tested whether exposing young HT29-MTX cells without mucus to the chitosan diluted in PBS acidified with HCl to pH 5.5 and at a concentration ranging from 1 to 5 mg/ml would be cytocompatible. Results shown in FIG. 4C suggest that none of the chitosan solutions affected the metabolic activity of the cells. Similarly, there was no sign of cytotoxicity of the highest chitosan concentration when tested on the mature and mucus-covered cells (FIG. 4D). Surprisingly, complementary analyses on the effect of chitosans on the cell membrane integrity did show an increase of cell membrane permeability to the red-fluorescent ethidium homodimer-1 dye. The effect was limited for the small DP8 chitosan, but more prominent for the larger DP52 and DP100 chitosans. However, the well documented biocompatibility of chitosan polymers and oligomers as well as the good cytocompatibility of the chitosan solutions encouraged us to further investigate whether they could be used as topical treatments to reinforce the natural barrier properties of the mucus layer.

### Example 5

### Chitosan Complexation can Reinforce the Barrier Properties of HT29-MTX Mucus

We then tested the ability of chitosan solutions to reinforce the barrier properties of mucus gel secreted by HT29-MTX cells. We first tested 4 kDa fluorescently labelled dextran molecules for a direct comparison with the in vitro reconstituted mucus system. Dextran is spontaneously taken up by HT29-MTX cells, thus the cell's total fluorescence can serve as an indicator of dextran's successful diffusion down to and across the cell layer. Flow cytometry analysis presented in FIG. 5A shows that there is less uptake of dextran by mucus-covered than by mucus-free cells. The decrease in median fluorescence confirms the presence of a protective mucus layer secreted by the cells. Complexation of DP8 chitosan to the mucus layer significantly decreased the median fluorescence compared to mucus-covered cells. However, neither DP52 nor DP100 chitosans led to significant changes in dextran diffusion and uptake by the cells.

We then tested whether chitosan complexation would affect the diffusion of a more chemically complex model molecule. The cholera toxin B subunit is a 12 kDa protein which has a strong affinity for the glucosphingolipid GM1 ganglioside of cell membranes and serves as a cell anchor to the full toxin. Cholera toxin B subunit has an estimated isoelectric point of 8.9, which means it will have a net positive charge at pH 5.5 used here and should have limited electrostatic interactions with the chitosan molecules. A fluorescent Cholera's toxin B (F-CTB) was used. Here again, the complexation of DP8 chitosan led to a decrease in median fluorescence of F-CTB when compared to young cells without mucus but also complexed with DP8 chitosan (FIG. 5B). However, similarly to dextran, there were no clear changes in cell uptake of the protein when the two chitosans of higher molecular weights were used to complex the mucus.

### Example 6

The smaller DP8 chitosan can reinforce the barrier properties of mucus in both the reconstituted and cell-based systems, although the effect was more pronounced in the cell system. The larger chitosans (i.e. DP100) were very effective in the reconstituted system but lost their effect when tested on the cell-based system. Such a difference in efficacy could stem from differences in adsorption of the chitosan onto the cell and mucus, but we found no difference in the quantities of chitosan complexed to the cell system between the three chitosan sizes (FIG. 6A). One can also hypothesise that such differences could arise from the more complex composition and structure of the HT29-MTX mucus, composed of multiple other proteins. Fluorescence confocal imaging of the complexed HT29-MTX cultured showed that the small DP8 chitosan distributed differently compared to the larger DP52 and DP100 chitosans. The DP8 chitosan was closer to the cells and seemed to diffuse in between cell multilayers to some extent, while in some areas DP52 and DP100 chitosans formed sheets that did not intermix with the cell layers. Here also, the complexation of chitosan with the secreted mucus as well as the sample preparation for microscopy may have removed the more loosely bound mucus layer. Hence it is likely that FIG. 6 depicts chitosans interacting with the tightly bound mucus layer and the glycocalyx. This ability of DP8 chitosan for a closer association around the cells and within the cell multilayer could explain its better blocking abilities in this system. Put together, these results clearly show the potential of reinforcing mucus layer with low molecular weight chitosans.

### Example 7

### Amino Acid Polymers

Effect of low or high molecular weight muco-adhesive polymers on mucin gels. A 4 μl drop of purified porcine gastric mucin was placed in a solution of poly-L-arginine (PLA, 1.9 kDa, 5 mg/mL), poly-L-lysine (PLL, 1.6 kDa, 5 mg/mL), or poly-L-lysine (PLL, 66 kDa, 5 mg/mL). The drop of mucin was observed under the microscope (10× objective). The three images are taken after 3 min, 12 min 30, and 50 min of contact. The small poly amino acid clearly crosslinks the mucin in depth, while the large poly-amino acid polymer changes the structure of the mucins drop and compacts it significantly.

### Example 8

### Penetration Test of Sperm Cells

A 4 μl drop of purified porcine gastric mucin complexed using a solution of chitosan (DP8, 5 mg/mL)(Figure 8A), or not complexed (FIG. 8B), were place in sperm solution. The images were recorded after 2 minutes. The complexed mucin had no sperm cell penetrating the outer shell, while the non-complexed mucins drop let sperm cells penetrate the gel.

Conclusions

It has thus been shown that small chitosan molecules (chito-oligosaccharides) can effectively bind and crosslink mucin and mucus gel, thereby retarding the diffusion of dextrans and cholera toxin B protein through the chitosan/mucin polyelectrolyte gel. The small chitosan polymers thus form a protective plug over the epithelial cells by complexing chitosan with mucin. Thus, they may reinforce the altered mucosa found in inflammatory bowel diseases or prevent ulcer-inducing Helicobacter bacteria from reaching the epithelium. Importantly, the biodegradability of chitosan and the normal turnover of mucus would make this modification only temporary, allowing other healing mechanisms to take place before its effects decreases. An important aspect of this invention is the use of relatively low molecular weight chitosans. Beyond the advantage of being able to diffuse through the mucus gel, LMW chitosan solutions have low viscosity and can have good solubility even at neutral pH. These leave a lot of possibilities to create effective mucosal treatment strategies through gels or sprays.

### REFERENCE LIST

1. Creeth, J. M. Constituents of mucus and their separation. Br. Med. Bull. 34, 17-24 (1978).

2. Linden, S. K., Sutton, P., Karlsson, N. G., Korolik, V. & McGuckin, M. A. Mucins in the mucosal barrier to infection. Mucosal Immunol. 1,183-197 (2008).

3. Gayton, J. L. Etiology, prevalence, and treatment of dry eye disease. Clin. Ophthalmol. 3,405-412 (2009).

4. Ying Joanna, N. D. & Thomson, W. M. Dry mouth—An overview. Singapore Dent. J. 36,12-17 (2015).

5. Colligris, B., Crooke, A., Huete-Toral, F. & Pintor, J. An update on dry eye disease molecular treatment: advances in drug pipelines. Expert Opin. Pharmacother. 15,1371-1390 (2014).

6. Shak, S., Capon, D. J., Hellmiss, R., Marsters, S. A. & Baker, C. L. Recombinant human DNase I reduces the viscosity of cystic fibrosis sputum. Proc. Natl. Acad. Sci. U. S. A. 87,9188-9192 (1990).

7. Pritchard, M. F. et al. A New Class of Safe Oligosaccharide Polymer Therapy To Modify the Mucus Barrier of Chronic Respiratory Disease. Mol. Pharm. (2016). doi:10.1021/acs.molpharmaceut.5b00794

8. Henke, M. O. & Ratjen, F. Mucolytics in cystic fibrosis. Paediatr. Respir. Rev. 8,24-29 (2007).

9. Ehre, C. et al. Overexpressing mouse model demonstrates the protective role of Muc5ac in the lungs. Proc. Natl. Acad. Sci. U. S. A. 109,16528-16533 (2012).

10. Gouyer, V. et al. Delivery of a mucin domain enriched in cysteine residues strengthens the intestinal mucous barrier. Sci. Rep. 5,9577 (2015).

11. Carrier, R. L. & Yildiz, H. M. Mucus strengthening formulations to alter mucus barrier properties. World Patent (2014).

12. Stremmel, W. et al. Mucosal protection by phosphatidylcholine. Dig. Dis. 30 Suppl 3,85-91 (2012).

13. Sun, J. et al. Therapeutic Potential to Modify the Mucus Barrier in Inflammatory Bowel Disease. Nutrients 8, (2016).

14. Willits, R. K. & Saltzman, W. M. The effect of synthetic polymers on the migration of monocytes through human cervical mucus. Biomaterials 25, 4563-4571 (2004).

15. Wang, Y.-Y. et al. Mucoadhesive nanoparticles may disrupt the protective human mucus barrier by altering its microstructure. PLoS One 6, e21547 (2011).

16. Chen, E. Y. T., Wang, Y.-C., Chen, C.-S. & Chin, W.-C. Functionalized positive nanoparticles reduce mucin swelling and dispersion. PLoS One 5, e15434 (2010).

17. Lai, S. K., Wang, Y.-Y., Cone, R., Wirtz, D. & Hanes, J. Altering Mucus Rheology to ‘Solidify’ Human Mucus at the Nanoscale. PLoS One 4, e4294 (2009).

18. Hillier, S. L. et al. In Vitro and In Vivo: The Story of Nonoxynol 9. JAIDS Journal of Acquired Immune Deficiency Syndromes 39, 1 (2005).

19. Dash, M., Chiellini, F., Ottenbrite, R. M. & Chiellini, E. Chitosan—A versatile semi-synthetic polymer in biomedical applications. Prog. Polym. Sci. 36, 981-1014 (2011/8).

20. Sogias, I. A., Williams, A. C. & Khutoryanskiy, V. V. Why is chitosan mucoadhesive? Biomacromolecules 9, 1837-1842 (2008).

21. Takeuchi, H. et al. Novel mucoadhesion tests for polymers and polymer-coated particles to design optimal mucoadhesive drug delivery systems. Adv. Drug Deliv. Rev. 57, 1583-1594 (2005).

22. Nikogeorgos, N., Efler, P., Basak Kayitmazer, A. & Lee, S. ‘Bio-glues’ to enhance slipperiness of mucins: improved lubricity and wear resistance of porcine gastric mucin (PGM) layers assisted by mucoadhesion with chitosan. Soft Matter 11, 489-498 (2014).

23. Deacon, M. P. et al. Atomic force microscopy of gastric mucin and chitosan mucoadhesive systems. Biochem. J 348 Pt 3, 557-563 (2000).

24. Celli, J. P. et al. Rheology of gastric mucin exhibits a pH-dependent sol-gel transition. Biomacromolecules 8, 1580-1586 (2007).

25. Navabi, N., McGuckin, M. A. & Lindén, S. K. Gastrointestinal cell lines form polarized epithelia with an adherent mucus layer when cultured in semi-wet interfaces with mechanical stimulation. PLoS One 8, e68761 (2013).

26. Orive, G. et al. Biocompatible oligochitosans as cationic modifiers of alginate/Ca microcapsules. J. Biomed. Mater. Res. B Appl. Biomater. 74, 429-439 (2005).

27. Baldrick, P. The safety of chitosan as a pharmaceutical excipient. Regul. Toxicol. Pharmacol. 56, 290-299 (2010).

28. Cuatrecasas, P. Interaction of Vibrio cholerae enterotoxin with cell membranes. Biochemistry 12, 3547-3558 (1973).

29. Boltin, D., Perets, T. T., Vilkin, A. & Niv, Y. Mucin function in inflammatory bowel disease: an update. J. Clin. Gastroenterol. 47, 106-111 (2013).

30. Bansil, R., Celli, J. P., Hardcastle, J. M. & Turner, B. S. The Influence of Mucus Microstructure and Rheology in Helicobacter pylori Infection. Front. Immunol. 4, 310 (2013).

31. Boucher, R. C. New concepts of the pathogenesis of cystic fibrosis lung disease. Eur. Respir. J. 23, 146-158 (2004).

32. Celli, J. et al. Viscoelastic properties and dynamics of porcine gastric mucin. Biomacromolecules 6, 1329-1333 (2005).

33. Kocevar-Nared, J., Kristl, J. & Smid-Korbar, J. Comparative rheological investigation of crude gastric mucin and natural gastric mucus. Biomaterials 18, 677-681 (1997).

34. Salim, E., Galais, A. & Trombotto, S. 4-(Hexyloxy)aniline-linked chitooligosaccharide-2,5-anhydro-D-mannofuranose. Molbank 2014, M815 (2014).

35. Qaqish, R. & Amiji, M. Synthesis of a fluorescent chitosan derivative and its application for the study of chitosan-mucin interactions. Carbohydr. Polym. 38, 99-107 (1999).

36. Serp, D., Mueller, M., Von Stockar, U. & Marison, I. W. Low-temperature electron microscopy for the study of polysaccharide ultrastructures in hydrogels. II. Effect of temperature on the structure of Ca2+-alginate beads. Biotechnol. Bioeng. 79, 253-259 (2002).

1. A composition comprising a mucoadhesive polymer and a physiological acceptable gelling agent, wherein said polymer consists of 4 to 20 monomer units linked to each other via ether, ester or amide bonds, which monomer units are selected from the list consisting of amino functionalised C6 sugars, C6 sugars, amino functionalised C5 sugars, C5 sugars, amino acids, fatty acid derivatised C6 sugars and fatty acid derivatised C5 sugars, wherein at least 50% of the monomer units of the mucoadhesive polymer comprise an amino group or at least 50% of the monomer units of the mucoadhesive polymer are selected from the list consisting of alanine, methionine, cysteine, phenylalanine, leucine, valine, isoleucine, fatty acid derivatised C6 sugars and fatty acid derivatised C5 sugars.

2. The composition according to claim 1, wherein the monomer unit is selected from the group consisting of arginine, lysine, histidine, ornithine, β-alanine, glycine, serine, threonine, asparagine, and glutamine.

3. The composition according to claim 1, wherein the mucoadhesive polymer is selected from the group consisting of poly-lysine, poly-orthinine, poly-arginine, cationised dialdehyde cellulose (DAC), cationic hydroxyethyl cellulose, chitosan, chitosan-trimethyl, chitosan-thioglycolic acid, chitosan-iminothiolane and chitosan-thioethylamidine.

4. The composition according to claim 1, wherein the mucoadhesive polymer is described by formula (I) 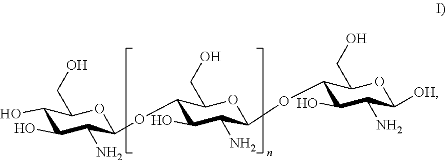
wherein, n is an integer between 2 and 10.

5. The composition according to claim 1, wherein the pharmaceutical acceptable carrier is water, DMSO, saline, or a combination thereof.

6. A composition comprising a mucoadhesive polymer and a physiologically acceptable gelling agent for use in therapy, the mucoadhesive polymer consisting of 4 to 20 monomer units linked to each other via ether, ester or amide bonds, which monomer units are selected from the list consisting of amino functionalised C6 sugars, C6 sugars, amino functionalised C5 sugars, C5 sugars, amino acids, fatty acid derivatised C6 sugars and fatty acid derivatised C5 sugars, wherein at least 50% of the monomer units of the mucoadhesive polymer comprise an amino group or at least 50% of the monomer units of the mucoadhesive polymer are selected from the list consisting of alanine, methionine, cysteine, phenylalanine, leucine, valine, isoleucine, fatty acid derivatised C6 sugars and fatty acid derivatised C5 sugars.

7. The composition for use in therapy according to claim 6, wherein the monomer unit is selected from the group consisting of arginine, lysine, histidine, ornithine, β-alanine, glycine, serine, threonine, asparagine, and glutamine.

8. The composition for use in therapy according to claim 6, wherein the mucoadhesive polymer is selected from the group consisting of poly-lysine, poly-orthinine, poly-arginine, cationised dialdehyde cellulose (DAC), cationic hydroxyethyl cellulose, chitosan, chitosan-trimethyl, chitosan-thioglycolic acid, chitosan-iminothiolane and chitosan-thioethylamidine.

9. The composition for use in therapy according to claim 6, wherein the mucoadhesive polymer is described by formula (I) 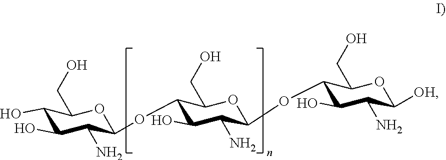
wherein, n is an integer between 2 and 10.

10. The composition for use in therapy according to claim 6, for the treatment of lesions of the mucous membrane.

11. A contraceptive composition comprising a mucoadhesive polymer and a physiological acceptable gelling agent, wherein the mucoadhesive polymer consists of 4 to 20 monomer units linked to each other via ether, ester or amide bonds, which monomer units are selected from the list consisting of amino functionalised C6 sugars, C6 sugars, amino functionalised C5 sugars, C5 sugars, amino acids, fatty acid derivatised C6 sugars and fatty acid derivatised C5 sugars, wherein at least 50% of the monomer units of the mucoadhesive polymer comprise an amino group or at least 50% of the monomer units of the mucoadhesive polymer are selected from the list consisting of alanine, methionine, cysteine, phenylalanine, leucine, valine, isoleucine, fatty acid derivatised C6 sugars and fatty acid derivatised C5 sugars.

12. A method comprising using of a mucoadhesive polymer consisting of 4 to 20 monomer units linked to each other via ether, ester or amide bonds, which monomer units are selected from the list consisting of amino functionalised C6 sugars, C6 sugars, amino functionalised C5 sugars, C5 sugars, amino acids, fatty acid derivatised C6 sugars and fatty acid derivatised C5 sugars, wherein at least 50% of the monomer units of the mucoadhesive polymer comprise an amino group or at least 50% of the monomer units of the mucoadhesive polymer are selected from the list consisting of alanine, methionine, cysteine, phenylalanine, leucine, valine, isoleucine, fatty acid derivatised C6 sugars and fatty acid derivatised C5 sugars, as a contraceptive.

13. A kit of parts comprising the composition according to claim 1, and an applicator.
